(19) United States
(12) Patent Application Publication
(10) Pub. No.: US 2024/0148294 A1
(43) Pub. Date: May 9, 2024

(54) Umbilical Cord Sensors and Methods of Using the Same

(71) Applicant: Johnson & Johnson Consumer Inc., Skillman, NJ (US)

(72) Inventors: Ming DONG, Hoboken, NJ (US); Richard A. INSEL, Rochester, NY (US)

(21) Appl. No.: 18/550,506

(22) PCT Filed: Mar. 9, 2022
(86) PCT No.: PCT/IB2022/052109

§ 371 Date: Sep. 14, 2023

Related U.S. Application Data
(60) Provisional application No. 63/162,133, filed on Mar. 17, 2021.

(51) Int. Cl. A61B 5/15; A61B 5/00; A61B 5/1455; A61B 5/1464

(52) U.S. Cl. CPC A61B 5/150038; A61B 5/14552; A61B 5/1464; A61B 5/150221; A61B 5/150984; A61B 5/4362; A61B 5/6838; A61B 5/6843; A61B 5/6882; A61B 5/6884; A61B 5/6885

(57) ABSTRACT

Umbilical cord sensors for monitoring biomarkers and other information associated with an umbilical cord are provided. Some exemplary embodiments of sensors include both an outer body and expandable inner body, with both bodies being disposed at least partially around an umbilical cord, the inner body being disposed within the outer body. The expandable inner body defines a receiving channel for receiving the umbilical cord, and is selectively expanded and contracted to engage and disengage with the umbilical cord. One or more biosensors and/or one or more sampling features can be included as part of the umbilical cord sensors, with the biosensors measuring one or more parameters and the sampling features obtaining one or more biomarkers associated with the umbilical cord. Exemplary methods of using umbilical cord sensors during the childbirth process are also provided, among other sensor configurations and methods of use.

### FIELD

The application relates generally to health monitoring devices and methods, and more particularly to umbilical cord sensors for monitoring various data, biofluids, biomarkers, and the like related to the health of a baby.

### BACKGROUND

Many diseases are of early-life origin. Early diagnosis of diseases, toxic exposures, effects, and susceptibilities in the still-developing body of infants may be required to develop successful intervention and treatment strategies to battle diseases. It is well documented that exposures to environmental chemical contaminants, including cigarette smoke constituents, for example, have adverse effects on fetal development and result in unfavorable health trajectories for affected children. Long-term outcomes such as diabetes, obesity, and chronic heart and kidney diseases have all been hypothesized or postulated to have their basis in fetal and childhood exposure and show an increased prevalence in children and newborns. It can be important to monitor fetal biofluids, biomarkers, and/or data associated therewith to help detect indications of various diseases, or susceptibility to various diseases for an unborn or just-born child. Such monitoring may also be helpful in assessing development of an unborn or just-born child, genotyping, and epigenetics, among other uses. For example, fetal biofluids, biomarkers, and/or data associated therewith may help provide useful information about the cognitive development of the unborn or just-born child.

During childbirth, after a baby is born, the umbilical cord is eventually clamped and cut. The timing of when to clamp and cut the cord has been the subject of debate. While there may be an ideal time range during which to clamp and cut the cord for a majority of babies, the optimal time for any one baby may be different than another. The time to clamp and cut the cord is more about whether a level of independence has been achieved by the baby than how much time has passed. For example, it may be desirable to wait to clamp and cut the cord until the lungs of the baby have been aerated, thereby allowing for a gentle switch from the oxygen-rich blood-flow that occurs from the placenta, through the umbilical cord, and to the baby to autonomous breathing and blood flow for the baby. This can help prevent a rapid increase in blood-pressure and sustain heart rate and cardiac output, thus supplying vital oxygen to the organs of the baby. Benefits to not immediately clamping and cutting the umbilical cord can include allowing the baby to receive a physiological transfer of oxygen-rich blood directly after birth during placental transfusion and, for preterm infants, reducing the risk of serious complications due to prematurity, such as anemia. However, there is at least some belief that failure to clamp and cut the umbilical cord in a timely manner may cause complications, such as increasing birth complications like neonatal respiratory distress and jaundice. Because each childbirth is a unique event, it may be useful to be able to monitor biomarkers and/or biofluids to help detect the ideal time to clamp and cut the umbilical cord on a case-by-case basis.

Accordingly, there is a need for sensors and sensing methods directed to monitoring biomarkers and/or biofluids associated with an umbilical cord. Such monitoring can provide the ability for early detection of diseases and disease indicators, information about possible disease susceptibility, child development, genotyping, and epigenetics, and information that can help assess the ideal time to clamp and cut an umbilical cord on a case-by-case basis, among other benefits.

### SUMMARY

Sensors and sensing methods for monitoring biomarkers and/or biofluids of the umbilical cord are disclosed herein. The sensors disclosed can provide the ability to monitor parameters and/or biomarkers associated with an outer surface of the umbilical cord, as well as such parameters and/or biomarkers associated with or otherwise disposed in the umbilical cord. The sensors include features that allow an umbilical cord sensor to be selectively attached and detached from the umbilical cord, and features to sense and/or sample biomarkers, biofluids, and the like. More specifically, the umbilical cord sensors of the present disclosure include outer bodies and expandable inner bodies, the expandable inner bodies allowing for the selective attachment and detachment of the sensors from the umbilical cord. Various features such as sensors, pulse oximeters, microneedles, and/or microchannels can be utilized to obtain the desired information from the umbilical cord. That information can then be used in a variety of contexts, including in real-time, for instance to assist in determining the ideal time to clamp and/or cut the umbilical cord for a particular baby, and to help gather data to help make assessments for the individual baby or for a collective group of babies. Such assessments may relate, for example, to disease detection, child development (e.g., cognitive development), genotyping, and epigenetics, among other uses.

One exemplary embodiment of an umbilical cord sensor includes an outer body and inner body disposed within the outer body, both bodies being configured to be disposed at least partially around an umbilical cord, and the inner body also being configured to be in contact with the umbilical cord (e.g., in an expanded configuration). A receiving channel is defined by the expandable inner body, with the receiving channel being configured to receive an umbilical cord. The sensor includes at least one of: (1) one or more biosensors; or (2) one or more sampling features. The biosensor(s) is structured and arranged to sense and/or measure one or more parameters associated with at least one of the umbilical cord or blood passing through the umbilical cord around which the outer body is at least partially disposed. The sampling feature(s) is structured and arranged to obtain one or more biomarkers associated with at least one of the umbilical cord or blood passing through the umbilical cord around which the outer body is at least partially disposed.

The outer body of the umbilical cord sensor can be configured to be disposed around a majority of a circumference of the umbilical cord around which it is disposed. As illustrated herein, this can result in the outer body being disposed around a majority of a circumference of a cross-section of a portion of the umbilical cord to which the umbilical cord sensor is disposed.

The umbilical cord sensor can include a two-way valve. The valve can be associated with the expandable inner body to allow fluid communication between the expandable inner body and a fluid source. As a result, a volume of the expandable inner body can be selectively expanded and contracted.

The umbilical cord sensor can include a pressure sensor. The pressure sensor can be associated with the expandable inner body and can be configured to assess at least one of an amount of pressure being exerted by the expandable inner body on the umbilical cord or an amount of pressure being experienced by the umbilical cord.

A variety of options can be used for the biosensor(s) and/or the sampling feature(s). For example, the sampling feature(s) can include at least one microchannel associated with the expandable inner body. The microchannel(s) can be in communication with at least one of the one or more biosensors or one or more separately disposed biosensors. As a result, one or more biomarkers associated with an outer surface of the umbilical cord can be analyzed by the respective one or more biosensors of the umbilical cord sensor or the one or more separately disposed biosensors. By way of further example, the sampling feature(s) can include at least one microneedle associated with the expandable inner body. The microneedle(s) can be configured to penetrate the umbilical cord to draw blood from the umbilical cord. Further, the microneedle(s) can be in communication with at least one of the one or more biosensors or one or more separately disposed biosensors such that one or more biomarkers associated with the drawn blood can be analyzed by the respective one or more biosensors of the umbilical cord sensor or the one or more separately disposed biosensors. By way of still a further example, the biosensor(s) can include at least one pulse oximeter associated with the expandable inner body. The pulse oximeter(s) can be configured to sense or measure one or more biomarkers associated with at least one of the umbilical cord or blood passing through the umbilical cord around which the outer body is at least partially disposed.

The umbilical cord sensor can include at least one cutting device. The cutting device(s) can be associated with at least one of the outer body and the expandable inner body. For example, the cutting device can be at least partially disposed in the outer body and configured to cut an umbilical cord disposed in the receiving channel. By way of further example, the cutting device can be at least partially disposed in the inner body and configured to cut an umbilical cord disposed in the receiving channel.

In some embodiments, the umbilical cord sensor can include a first arm, a second arm opposed to the first arm, and teeth. More specifically, first teeth can be associated with the first arm and/or a portion of the expandable inner body disposed within the first arm, and second teeth can be associated with the second arm and/or a portion of the expandable inner body disposed within the second arm. The first and second teeth can be opposed to each other and configured to clamp the umbilical cord around which the outer body is at least partially disposed.

One exemplary method of monitoring one or more biomarkers includes disposing an umbilical cord sensor on an umbilical cord such that the sensor is disposed around at least a portion of the umbilical cord. The method further includes at least one of: (1) sensing or measuring one or more parameters associated with one or more biomarkers of at least one of the umbilical cord or blood passing through the umbilical cord around which the umbilical cord sensor is disposed; or (2) obtaining a sample of one or more biomarkers associated with at least one of the umbilical cord or blood passing through the umbilical cord around which the umbilical cord is disposed.

The method can also include expanding an expandable body of the umbilical cord sensor to assist in securing the umbilical cord sensor to the umbilical cord. In some such embodiments, expanding the expandable body can include using at least one pressure sensor associated with the umbilical cord sensor to assess at least one of an amount of pressure being exerted by the expandable body on the umbilical cord or an amount of pressure being experienced by the umbilical cord. Alternatively, or additionally, expanding the expandable body can include operating a two-way valve to expand the expandable body.

Disposing the umbilical cord sensor on an umbilical cord sensor can include disposing the umbilical cord sensor around a majority of a circumference of a cross-section of a portion of the umbilical cord to which the umbilical cord sensor is disposed. In some embodiments, the method can include clamping the umbilical cord with the umbilical cord sensor. The method can also include cutting the umbilical cord. The cutting can be done by an outside cutting device, or alternatively, it can be done using a cutting device(s) of the umbilical cord sensor.

The action of obtaining a sample of one or more biomarkers associated with at least one of the umbilical cord or blood passing through the umbilical cord around which the umbilical cord is disposed can include obtaining one or more biomarkers associated with an outer surface of the umbilical cord. Alternatively, or additionally, the action of obtaining a sample of one or more biomarkers associated with at least one of the umbilical cord or blood passing through the umbilical cord around which the umbilical cord is disposed can include drawing blood from the umbilical cord sensor.

The action of sensing or measuring one or more parameters associated with one or more biomarkers of at least one of the umbilical cord or blood passing through the umbilical cord around which the umbilical cord sensor is disposed can include sensing or measuring using at least one pulse oximeter associated with the umbilical cord sensor.

The one or more biomarkers sensed, measured, and/or sampled can be one or more biomarkers associated with at least one of the umbilical cord or a placenta. In some embodiments, the method can further include assessing a medical condition of at least one of a fetus, a baby, or a mother based on the one or more biomarkers. Alternatively, or additionally, in some embodiment the method can include monitoring development of at least one of a fetus, a baby, or a mother based on the one or more biomarkers. The development can include, but is not limited to, cognitive development of the fetus or baby.

### BRIEF DESCRIPTION OF DRAWINGS

This disclosure will be more fully understood from the following detailed description taken in conjunction with the accompanying drawings, in which:

FIG. 1 is a schematic perspective view of one exemplary embodiment of an umbilical cord sensor disposed around an umbilical cord;

FIG. 2A is a cross-sectional front view of the umbilical cord sensor of FIG. 1 taken along line A-A with an inner body of the sensor being in an unexpanded configuration;

FIG. 2B is the cross-sectional front view of FIG. 2A with the inner body of the sensor in a first expanded configuration;

FIG. 3 is a cross-sectional front view of an alternative exemplary embodiment of an umbilical cord sensor;

FIG. 4 is a cross-sectional front view of a further alternative embodiment of an umbilical cord sensor;

FIG. 5A is a cross-sectional front view of still a further alternative exemplary embodiment of an umbilical cord sensor, in this instance with an inner body of the sensor being in a second expanded configuration, sometimes referred to as a clamped configuration;

FIG. 5B is the cross-sectional front view of FIG. 5A illustrating a cutting component of the sensor; and

FIG. 6 is a top view of yet a further alternative embodiment of an umbilical cord sensor.

### DETAILED DESCRIPTION

Certain exemplary embodiments will now be described to provide an overall understanding of the principles of the structure, function, manufacture, and use of the devices (e.g., sensors) and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. Further, in the present disclosure, like-numbered components of the embodiments generally have similar features. Additionally, to the extent that linear or circular dimensions are used in the description of the disclosed devices and methods, such dimensions are not intended to limit the types of shapes that can be used in conjunction with such devices and methods. A person skilled in the art will recognize that an equivalent to such linear and circular dimensions can easily be determined for any geometric shape. Sizes and shapes of the devices, and the components thereof, can depend at least on the anatomy of the subject in which the devices will be used, the size and shape of components with which the devices will be used, and the methods and procedures in which the devices will be used.

The figures provided herein are not necessarily to scale. Further, at least some of the embodiments illustrated herein are schematic in nature such that, in practice, some of the components of the embodiment may respond differently than illustrated in practice. By way of non-limiting example, the disclosure provides for an expandable inner body, and while in the illustrated embodiments the expandable inner body typically remains confined to locations bound by the outer body, in practice the expandable inner body may expand beyond such confines and extend past and/or around portions of the outer body and/or around portions of an umbilical cord not encircled by the outer body. Further, a number of terms may be used throughout the disclosure interchangeably, even if they do not have the same meaning, but will be understood by a person skilled in the art. By way of non-limiting example, the terms “biomarker” and “biofluid” may be used interchangeably.

The present disclosure is generally directed to umbilical cord sensors that can be used to help monitor various parameters and/or data associated with an umbilical cord. The umbilical cord sensor can be placed on the umbilical cord during childbirth and can: (1) sense or measure one or more parameters associated with the umbilical cord and/or blood passing through the umbilical cord; and/or (2) obtain one or more biomarkers associated with the umbilical cord and/or blood passing through the umbilical cord. The data, information, and the like obtained by the umbilical cord sensor, in turn, can be used both to make initial assessments directly related to the childbirth process, such as helping to determine when to clamp and/or cut the umbilical cord, and/or used to detect and/or analyze development of the newborn baby, such as helping to monitor for early detection of diseases, disease indicators, and/or disease susceptibility, as well as evaluating child development (e.g., cognitive development), genotyping, and/or epigenetics.

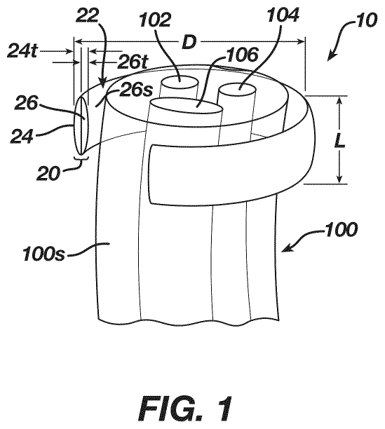

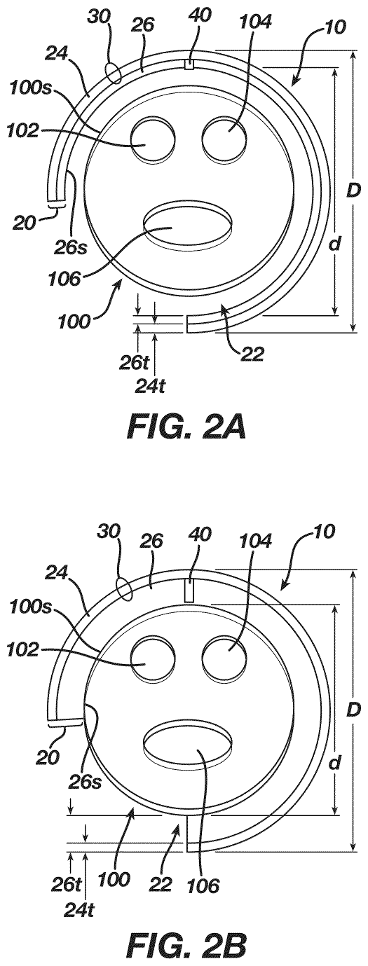

FIGS. 1-2B illustrates one exemplary embodiment of an umbilical cord sensor 10. The sensor 10 includes a body 20 that defines a receiving channel 22 through which an umbilical cord 100 can be disposed and then one or both of: (1) one or more biosensors or other sensing devices to sense or measure one or more parameters associated with the umbilical cord 100 and/or blood passing through the umbilical cord 100; or (2) one or more sampling features to obtain biomarkers and/or other data that can be used for various medical purposes, such as monitoring for early detection of diseases, disease indicators, and/or disease susceptibility, evaluating child development (e.g., cognitive development), genotyping, epigenetics, and/or assessing the ideal time to clamp and cut the umbilical cord 100. As shown, the body 20 can include both an outer body 24 and an inner body 26, with the inner body being disposed within the outer body.

The outer body 24, sometimes referred to as a housing, can be a substantially rigid, cylindrical or ring-shaped structure that is configured to be disposed at least partially around a portion of the umbilical cord 100. In the illustrated embodiment of FIGS. 1-2B, the outer body 24 is disposed around approximately three-quarters of the circumference of the umbilical cord 100, which is shown in reference to a cross-section of a portion of the umbilical cord 100 to which the umbilical cord sensor 10 is disposed. Not disposing the outer body 24 fully around the circumference of the umbilical cord 100 can help make it easier to slide or otherwise dispose the sensor 10 onto and/or around the umbilical cord 100. It can also provide some additional relief for the expandable inner body 26 by providing an outlet for the inner body 26 to over-expand into if desired, such expansion described in greater detail below. In other configurations the outer body 24 can be disposed around a majority of the circumference of the umbilical cord 100 (i.e., just over half of the circumference of the umbilical cord), over greater than three-quarters of the circumference of the umbilical cord 100, and up to disposed fully around the circumference of the umbilical cord 100. The outer body 24 can be disposed around any portion of the circumference of the umbilical cord 100 provided that the sensor 10 is configured to be positioned, and likely secured, at a desired location along a length of the umbilical cord 100. Even configurations that do not extend around half or more of the circumference of the umbilical cord 100 are possible, although such embodiments are more likely to include features that help secure the location of the sensor 10 with respect to the umbilical cord 100 to prevent the sensor 10 from falling off the umbilical cord 100.

As discussed below, the inner body 26 can help secure the sensor 10 with respect to umbilical cord 100 to selectively fix a location of the sensor 10 (e.g., a desired location) to the umbilical cord 100. Other features to secure the location of the sensor 10 with respect to the umbilical cord 100 can also be used, in lieu of or in addition to the inner body 26, such as teeth located on an outer and/or inner body and/or one or more pins or other components that can extend from one or both of the outer and inner bodies and engage the umbilical cord to position and secure the sensor relative to the umbilical cord.

The rigid nature of the outer body 24 can help provide a structure against which the inner body 26 can expand. With that said, in other embodiments the outer body 24 can be semi-rigid and/or can be configured to expand if such expansion would not cause undesirable damage to any surrounding anatomy (if any; usually there will not be any surrounding anatomy if the sensor 10 is attached to the umbilical cord 100 outside of the mother, which is the most discussed and likely common use, but not necessarily the only use). In some embodiments, the outer and inner bodies 24, 26 may be a singular body capable of expanding such that the outer and inner bodies are one and the same. Some exemplary materials from which the outer body 24 can be made include but are not limited to titanium, aluminum, stainless steel, plastics, and other metals or thermoformed materials.

In the illustrated embodiment, the outer body 24 has a ring-shape that can also be considered cylindrical. A diameter D of the outer body 24 can be approximately in the range of about 2 centimeters to about 3 centimeters, and in some embodiments the diameter can be approximately 2.5 centimeters (i.e., approximately one inch). A length L of the outer body 24 can be approximately in the range of about 0.1 centimeters to about 8 centimeters, and in some embodiments the length can be approximately 0.5 centimeters. Other shapes, sizes and configurations are possible without departing from the spirit of the present disclosure.

The inner body 26 can be an expandable structure, sometimes referred to as a bladder, disposed within the outer body 24 and configured to be disposed at least partially around a portion of the umbilical cord 100. In the illustrated embodiment of FIGS. 1-2B, the inner body 26 is disposed around approximately three-quarters of the circumference of the umbilical cord 100. Similar to the outer body 24, in other embodiments the inner body 26 can be configured to be disposed around more or less of the umbilical cord 100 than illustrated. An inner surface 26s of the inner body 26 defines a circumference of the receiving channel 22 of the sensor 100, such circumference being definable even when the inner body 26 is not a full circle. In such instances, the size of the circumference of the receiving channel 22 can either be based on the existing length/circumference of the inner surface 26s, or the remainder of the circumferences can be extrapolated from the relevant dimensions of the inner body 26. As the inner body 26 expands and contracts, the inner surface 26 of the inner body 26 moves radially inwards and outwards, thereby changing a diameter of the receiving channel 22. As shown, the receiving channel 22 receives the umbilical cord 100.

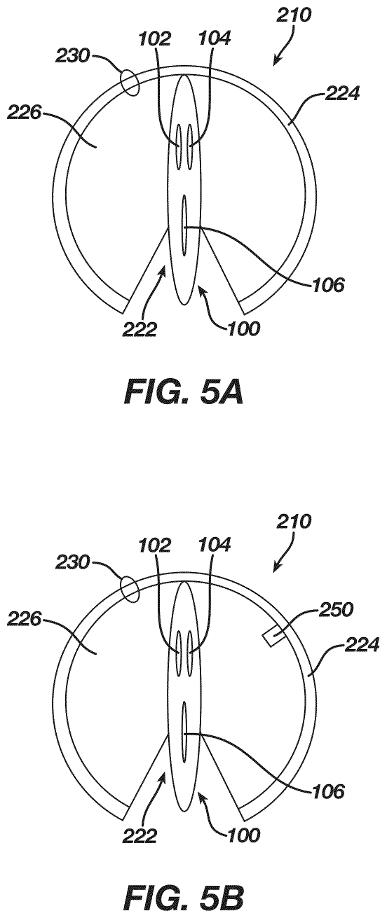

The inner body 26 is expandable, which can help secure the sensor 10 with respect to umbilical cord 100 to selectively fix a location of the sensor 10 (e.g., a desired location) to the umbilical cord 100. In FIGS. 1 and 2A, the inner body 26 is in an unexpanded configuration, sometimes referred to as a contracted configuration or an initial configuration, having a thickness 26t similar to a thickness 24t of the outer body 24 (although the thicknesses do not have to be similar), while in FIG. 2B the inner body is in an expanded, engaged configuration, also referred to herein as a first expanded configuration. Further expansion, to a second expanded configuration, is described below with respect to an umbilical cord sensor shown in FIGS. 5A and 5B. Expansion of the inner body 26 causes the thickness 26t to increase.

As the expandable inner body 26 expands, its volume increases and the inner surface 26s that defines a diameter d of the channel 22 decreases. In the first expanded configuration, the inner body 26 is expanded such that it engages an outer surface 100s of the umbilical cord 100 to help fix a location of the sensor 10 with respect to the umbilical cord 100. In some instances, the engagement can be tight such that the sensor 10 will generally not be able to slide along a length of the umbilical cord 100 when the inner body 26 is in the first expanded configuration, while in other instances the engagement can be a little looser, still generally securing the sensor 10 to the umbilical cord 100, but allowing for at least some sliding movement of the sensor 10 along a length of the umbilical cord 100. Typically in the first expanded configuration the inner body 26 is not so tight around the umbilical cord 100 so as to clamp the umbilical cord 100. As a result, blood can typically continue to flow through the arteries 102, 104 and the vein 106 of the umbilical cord 100 when the inner body 26 is in the first expanded configuration. The inner body 26 is also contractable, meaning that the expansion of the inner body 26 can be reduced, allowing the inner body 26 to return to the contracted configuration shown in FIGS. 1 and 2A.

Any variety of techniques known for selectively expanding and contracting an expandable structure can be employed to expand and contract the inner body 26. In the illustrated embodiment, a two-way valve 30 is provided that allows fluid to be selective added and removed from the expandable inner body 26. For example, a fluid source (not shown) can be provided, such as from a component separate from the sensor 10, and can be in fluid communication with the expandable inner body 26 to selectively expand and contract a volume of the inner body 26. In other embodiments, the fluid source can be disposed within and/or on the umbilical cord sensor 10. By way of non-limiting example, the fluid source can be disposed within and/or on the outer body 24, with the two-way valve 30 allowing for communication between the fluid source of the outer body 24 and the expandable inner body 26.

Any number of fluids can be disposed in or otherwise associated with the fluid source and used to selectively expand the inner body 26. For example, air can be disposed in a chamber of a fluid source and selectively added and removed from the inner body 26 by way of the two-way valve 30. In alternative embodiments, the air can be from an outside environment such that the fluid source is the outside environment and is not “disposed in” a fluid source. It is possible that other fluids can be used to expand the inner body 26.

The flow of the fluid between the fluid source and the expandable inner body 26 can be controlled using a variety of techniques. For example, the two-way valve 30 can be configured to operate based on a sensed pressure between the expandable inner body 26 and the umbilical cord 100, such as blood pressure. The sensed pressure can inform the two-way valve whether to introduce or remove fluid from the expandable inner body 26 to achieve the desired fit of the sensor 10 around the umbilical cord 100. By way of further example, the two-way valve 30 can be manually operated such that a doctor, nurse, or other person(s) involved in the procedure can control when fluid enters and exits the expandable inner body 26. Decisions about whether to expand or contract can be informed by visual inspection, instructions from others, and/or based on feedback from umbilical cord sensor 10 and related components. For example, in some embodiments, the two-way valve 30 can be manually activated by the doctor, nurse, or other person(s) involved in the procedure for purposes of coupling the umbilical cord sensor 10 to the umbilical cord 100 or de-coupling the umbilical cord sensor 10 from the umbilical cord 100. That is, the two-way valve 30 can be activated to fill the inner body 26 sufficiently full of fluid to position and maintain the location of the umbilical cord sensor 10 at a desired location on the umbilical cord 100. Likewise, the two-way valve 30 can be activated to remove fluid from the inner body 26 to allow the position of the umbilical cord sensor 10 to be moved relative to the umbilical cord 100 and/or removed from the umbilical cord 100.

Any number of materials can be used to form the expandable inner body 26, including but not limited to rubber, neoprene, thermoplastic elastomer (TPE), etc. Further, a size and shape of the inner body 26 can generally be akin to, or at least compatible with, a size and shape of the outer body 24. A volume of the inner body 26 in the unexpanded configuration can be, by way of non-limiting example, approximately in the range of about 0.2 milliliters to about 0.4 milliliters, and in some embodiments it can be about 0.3 milliliters, and a volume of the inner body 26 in the first expanded configuration can be, by way of non-limiting example, approximately in the range of about 0.5 milliliters to about 2.5 milliliters, and in some embodiments it can be about 1.5 milliliters. Factors that impact the size and shape of the inner body 26 can depend, at least in part, on the size and shape of the outer body 24 and the size of the umbilical cord 100. Like the outer body 24, a variety of shapes, sizes, and configurations are possible for the inner body 26.

As described below, one or more sensors (e.g., biosensors) and/or sampling features can be included as part of the umbilical cord sensor 10. The data and/or parameters measured by such sensor(s) and feature(s) can provide information to the person controlling the two-way control valve 30 about whether expansion or contraction, or further expansion or contraction, is desired. In other embodiments, the two-way valve 30 can be automated such that feedback from the one or more sensors (e.g., biosensors), sampling features, and/or other data gathering components or information more generally can help inform a computer processor to selectively turn on and off fluid flow between the fluid source and the expandable inner body 26, in either direction, based on the feedback. Accordingly, by way of non-limiting example, the computer processor can instruct the two-way valve 30 to allow fluid flow from the fluid source, into the expandable inner body 26, until a certain percentage of the inner surface 26s is in contact with the umbilical cord 100, after which the two-way valve 30 is closed to stop the fluid flow. A person skilled in the art, in view of the present disclosures, will appreciate other parameters, data, etc. that can be relied upon to inform manual or automated operation of the two-way valve 30. The computer processor (not shown) can be part of a computer chip (not shown) or the like disposed on the umbilical cord sensor 10, the fluid source, and/or an outside component that is in communication with one or more of the sensor 10, the fluid source, and the umbilical cord 100.

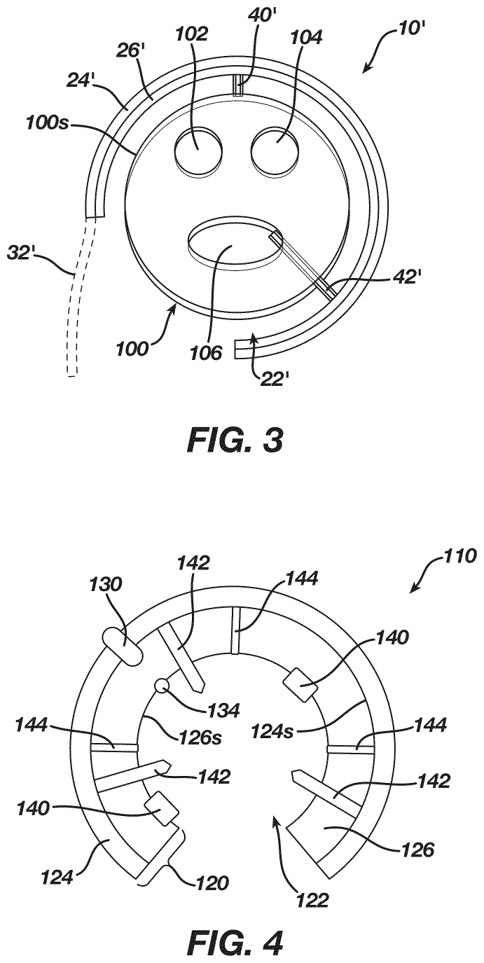

As discussed above, in some embodiments an umbilical cord sensor 10′ can be disposed around the entire circumference of the umbilical cord 100. In some such embodiments, one or more features can be included with an outer body 24′ to help dispose the sensor 10′ around the umbilical cord 100 at least because there is no easy way to slide the sensor 10′ on or off from either end of the umbilical cord 100. For example, and as shown schematically with respect to the umbilical cord sensor 10′ of FIG. 3, a hinged door 32′ can be provided as part of the outer body 24′, the door 32′ being able to opened to allow the umbilical cord sensor 10′ to be slid onto the umbilical cord 100, and when closed can prevent the sensor 10′ from falling off the umbilical cord 100. In the illustrated embodiment, an inner body 26′ is not included as part of the hinged door 32′, but in other configurations the sensor can be designed in a manner that would allow the inner body to be part of the hinged door as well, whether as a second, separate inner body, or as part of a singular inner body. In other embodiments, the hinged door 32′ can more generally be a second piece of the outer body 24′ that can be selectively coupled and de-coupled from the main portion of the outer body 24′. Such selective coupling can be achieved using any known techniques for coupling one component to another, including various forms of mechanical engagement, such as male-female coupling mechanisms, interference fits, snap-fits, stretch-fits, and/or threaded configurations, among others.

Turning back to FIGS. 1-2B, the umbilical cord sensor 10 can include one or both of: (1) one or more biosensors, or sensing devices (use of one term does not preclude the other); or (2) one or more sampling features. In some instances, a biosensor(s) can be integrated with a sampling feature(s) such that they are one and the same. For example, as discussed below, some biosensors sense biomarkers, which can be considered a form of sampling. Biosensors of the present disclosure are structured and/or arranged to sense and/or measure one or more parameters associated with at least one of the umbilical cord 100 or blood passing through the umbilical cord 100 disposed in the receiving channel 22, and thus around which the outer body 24 of the sensor 10 is at least partially disposed. Sampling features of the present disclosure are structured and/or arranged to obtain one or more biomarkers associated with at least one of the umbilical cord 100 or blood passing through the umbilical cord 100 disposed in the receiving channel 22, and thus around which the outer body 24 of the sensor 10 is at least partially disposed. A sampling feature can be considered any component of the umbilical cord sensor that is capable of obtaining a sample from any portion of the umbilical cord, whether that sample remains with the umbilical cord or is subsequently removed from the umbilical cord.

The present disclosure provides for some non-limiting examples of biosensors that can be used in conjunction with monitoring various parameters associated with the umbilical cord, including but not limited to blood passing through the umbilical cord. The present disclosure likewise provides for some non-limiting examples of sampling features that can be used to obtain biomarkers, biofluids, and/or other data that can be used for various medical purposes, including but not limited to monitoring for early detection of diseases, disease indicators, and/or disease susceptibility, evaluating child development (e.g., cognitive development), genotyping, epigenetics, and/or assessing the ideal time to clamp and cut the umbilical cord. Accordingly, while the illustrated embodiments, and descriptions herein, provide some exemplary examples of such biosensors and sampling features, such disclosures are by no means limiting in terms of the type, configuration, placement, and/or use of such biosensors and/or sampling features.

FIGS. 2A and 2B illustrate a biosensor 40 that is associated with the inner body 26 such that it can sense blood flow through the umbilical cord 100, such as by electrical impedance or a photodiode. As shown, the biosensor 40 is coupled to the outer body 24, extending from an inner surface 24s thereof, and can move with the inner body 26 (e.g., expand) as the inner body 26 is expanded from the unexpanded configuration of FIG. 2A to the first expanded configuration of FIG. 2B. In other embodiments the biosensor 40 can be disposed within or otherwise associated with an inner surface 26s of the inner body 26 such that as the inner body 26 expands, the biosensor 40 moves with the inner surface 26s, i.e., not necessarily expanding as shown in FIG. 2B. When the biosensor 40 is in contact with the umbilical cord 100, it can sense blood flow. Other biosensors may be used that do not necessarily require direct contact with the umbilical cord 100 to detect blood flow.

FIG. 3 illustrates an umbilical cord sensor 10′ that is similar to the umbilical cord sensor 10 of FIGS. 1-2B but includes alternative, non-limiting examples of biosensors. Similar to the umbilical cord sensor 10, the umbilical cord sensor 10′ includes an outer body 24′ and an expandable inner body 26′, the expandable inner body 26′ defining a receiving channel 22′ configured to receive the umbilical cord 100. In the illustrated embodiment, the sensor 10′ includes two biosensors 40′, 42′ associated with the inner body 26′. The first biosensor 40′ is configured such that it can sense (typically, though not necessarily, through contact with the surface 100s of the umbilical cord 100) various parameters or data associated with a surface 100s of the umbilical cord 100. In the illustrated embodiment, the first biosensor 40′ can sense one or more biomarkers on the surface 100s of the umbilical cord 100. As shown, it can be done even when the inner body 26′ is in the unexpanded configuration. As the inner body 26′ expands, such as to the first expanded configuration, the first biosensor 40′ can remain engaged with the surface 100s of the umbilical cord 100. In other embodiments, like the sensor 10 of FIGS. 2A and 2B, the sensor 10 can be configured to move in conjunction with the expansion of the inner body 26.

The second biosensor 42′ is configured such that it can sense various parameters or data associated with the inside of the umbilical cord 100. This is typically done through contacting an internal portion of the umbilical cord 100. In the illustrated embodiment the second biosensor 42′ is configured to access the umbilical vein 106, thereby allowing it to sense one or more biomarkers associated with the vein 106. In other embodiments, the second biosensor 42′ can be configured to access one of the umbilical arteries 102, 104 and/or additional biosensors can be used to access more than one of the arteries 102, 104 and vein 106. Still further, biosensors can be configured to access other portions of the umbilical cord 100 if doing so is advantageous for obtaining information that would be helpful in achieving the purposes described herein. The first and second biosensors 40′, 42′ can alternatively be considered as sampling features at least because they sense, or otherwise sample, biomarkers.

FIG. 4 illustrates another exemplary embodiment of an umbilical cord sensor 110. Similar to the sensors 10, 10′ of FIGS. 1-3, the umbilical cord sensor 110 includes an outer body 124 and an expandable inner body 126 (collectively a body 120), the expandable inner body 126 defining a receiving channel 122 configured to receive an umbilical cord. In the illustrated embodiment, the expandable inner body 126 is in a first expanded configuration, similar to the same configuration described above with respect to the umbilical cord sensor 10 of FIG. 2B. Each of the outer and inner bodies 124, 126 is configured to extend at least partially around an umbilical cord that can be received by the receiving channel 122, as shown each body 124, 126 having a circumference that exceeds three-quarters of the circumference of an umbilical cord received by the receiving channel 122, forming a “C-shape.” Similar to the earlier descriptions of circumferences with respect to the sensor 10, the circumferences of the inner and outer bodies 124, 126 can be define even when the bodies 124, 126 are not a full circle.

Also similar to the sensors 10, 10′ of FIGS. 1-3, the umbilical cord sensor 110 can include a two-way valve 130 to help selectively inflate and deflate the inner body 126, and one or both of: (1) one or more biosensors, or sensing devices; or (2) one or more sampling features. The illustrated embodiment provides for some such sensors and sampling features that are similar to those described above with respect to FIGS. 1-3, others that are variations of such sensors and sampling features, and yet others that are newly illustrated in FIG. 4. A person skilled in the art, in view of the present disclosures, will understand how to incorporate these various options for sensors and sampling features, including particular features of such sensors and sampling features, across any of the umbilical cord sensor embodiments provided for herein or otherwise derivable from the present disclosures. The various sensors and sampling features provided for herein can be mixed and matched as desired. Other sensors and sampling features can also be incorporated into any of the umbilical cord sensors provided for herein, or other umbilical cord sensors derivable from the present disclosure.

The umbilical cord sensor 110 can include one or more sensing devices configured to sense or measure one or more parameters, and/or obtain samples of one or more biomarkers, associated with an outside surface of an umbilical cord (not shown). As shown, two pulse oximeters 140 are disposed in, coupled to, or are otherwise associated with the inner body 126. Other amounts of pulse oximeters 140, including one or more than two, can be used, with a person skilled in the art recognizing how to utilize data from multiple pulse oximeters when more than one is employed. The pulse oximeters 140 can measure the blood flow rate through the umbilical cord. This can help monitor when blood flow through the cord is reducing or stops, such as when the lungs of the newborn baby begin to function. In some embodiments, as a reduction in blood flow is sensed, a computer processor, such as the one described above, can instruct the inner body 126 to expand to clamp and/or close off the cord. In some embodiments, based on the feedback from the pulse oximeter(s) 140, one or more signals (e.g., audio, visual, etc.) can be provided to the doctor, nurse, or other person(s) involved in the procedure that it is safe to cut the umbilical cord due to blood flow having stopped, or at least been sufficiently reduced. Alternatively, or additionally, the computer processor can also instruct a cutting device incorporated with the umbilical cord sensor, such as a cutting device 250 illustrated in FIG. 5B, described below, to cut the cord based on feedback from the pulse oximeter(s) 140. Relatedly, if the feedback from the pulse oximeter(s) 140 is that blood flow has not sufficiently reduced, such as if the lungs of the newborn baby are not functioning properly enough to take over providing oxygen to the newborn baby, such feedback can trigger one or more signals (e.g., audio, visual, etc.) that are different than the signal(s) provided for when blood flow has been sufficiently reduced or stopped to allow for the umbilical cord to be cut, so the doctor, nurse, or other person(s) involved in the procedure know that the umbilical cord should not be cut.

Another sensor provided for in conjunction with the inner body 126 is a pressure sensor 134. As shown, the pressure sensor 134 can be disposed at an inner surface 126s of the inner body 126, allowing it to detect the pressure being applied by the inner body 126, and thus the umbilical cord sensor 110, on the umbilical cord and/or pressure being experienced by the umbilical cord. The pressure sensor 134 can provide feedback, such as by way of the computer processor or otherwise, to the two-way valve 130 to help regulate an amount of fluid in the inner body 126 to alter the amount of pressure being applied to the umbilical cord by the umbilical cord sensor 110. This can help insure, for example, that the umbilical cord sensor 110 is not being undesirably clamped at a particular time.

The illustrated embodiment also includes three sampling features configured to sense biomarkers and/or biofluids inside the umbilical cord, such as in the arteries and vein of the cord. In the illustrated embodiment, each sampling feature is a microneedle 142 that extends from an inner surface 124s of the outer body 124, through the inner body 126, and extends radially inward from the inner body 126 in a deployed configuration such that it can provide access inside of an umbilical cord disposed in the receiving channel 122. As provided for herein, such microneedles 142, or biosensors or sampling features more generally, can be configured to be associated with just the inner body 126 as well, among other configurations provided for herein or otherwise derivable from the present disclosures. The illustrated microneedles 142 can be actuated and puncture or otherwise penetrate the cord, for example after the cord has been clamped off. The action of actuating the microneedles 142 can be automated such that they actuate towards and into the umbilical cord 100 once the sensor 10 is secured on the cord 100. In some embodiments, the microneedles 142 can include one or more pressure sensors (not shown), for example at a distal tip of one or more of the microneedles 142. As the microneedle(s) 142 punctures through the umbilical cord 100 (often slowly), the sensor(s) can sense pressure against the cord 100 and stop advancement of the microneedle(s) 142 for further penetration into the cord 100. For example, a stop order may be communicated such that the microneedle(s) 142 stops once a threshold pressure drop is identified, such pressure drop being indicative of the microneedle(s) 142 reaching an artery or vein.

In some embodiments, the microneedle(s) 142 can include channels, including but not limited to microfluidic channels, to collect and sense biomarkers on the inside of the cord, including in blood in the cord. A person skilled in the art, in view of the present disclosures, will understand that the illustrated microneedles 142 can also perform functions of biosensors, such as by sensing various parameters associated with the umbilical cord. In some embodiments, one or more microneedles 142 can be in communication with one or more biosensors associated with the sensor 110 and/or one or more biosensors that are separately disposed from the sensor 110. This can allow biomarkers associated with drawn blood to be analyzed by one or more biosensors of the umbilical cord sensor 110 and/or one or more biosensors separately disposed from the sensor 110.

Still further, the illustrated embodiment includes three sampling features configured to sense biomarkers on a surface of the umbilical cord. In the illustrated embodiment, each sampling feature is a microchannel 144 that extends from the inner surface 124s of the outer body 124, through the inner body 126, and terminates at, or at least proximate to, the inner surface 126s of the inner body 126 such that the microchannel 144 can access the outside surface of the umbilical cord to sample biomarkers from the same. A person skilled in the art, in view of the present disclosures, will understand that the illustrated microchannels 144 can also perform functions of biosensors, such as by sensing various parameters associated with the umbilical cord. Non-limiting examples of such configurations are described above with respect to the umbilical cord sensors of FIGS. 1-3. In some embodiments, one or more microchannels 144 can be in communication with one or more biosensors associated with the sensor 110 and/or one or more biosensors that are separately disposed from the sensor 110. This can allow biomarkers associated with drawn blood to be analyzed by one or more biosensors of the umbilical cord sensor 110 and/or one or more biosensors separately disposed from the sensor 110.

To the extent various sampling features (e.g., microneedles 142, microchannels 144) illustrated herein appear to terminate at a particular location without providing the ability to easily access the samples taken, a person skilled in the art will understand how to configure the umbilical cord sensor 110, and its various features, to allow the samples to be accessed. There are a variety of ways by which samples can be collected and accessed, including but not limited to providing outlets at the end of the sampling features to collect samples and/or including features within the umbilical cord sensor 110 that can analyze the biomarkers directly within the umbilical cord sensor 110 without removing the biomarker from the umbilical cord and/or the umbilical cord sensor 110. By way of non-limiting example, one instance in which real-time analysis of biomarkers can be performed within the umbilical cord sensor includes analyzing lactate in cord blood for predictions of hypoxic-ischemic encephalopathy (HIE), thus allowing for immediate medical interventions at birth. By way of further non-limiting example, another instance in which real-time analysis of biomarkers can be performed within the umbilical cord sensor includes analyzing acute phase reactant biomarkers such as C-reactive protein, serum amyloid A, haptoglobin, serum amyloid P, and/or ferritin, which can help predict early onset of neonatal sepsis, and thus allowing for early intervention related to the same.

Further, as indicated, the sensors and sampling features provided for in the illustrated embodiments are non-limiting examples of such sensors and sampling features. Other sensors and/or sampling features that can be incorporated into the umbilical cord sensor include, but are not limited to: microneedles that automatically eject from the umbilical cord sensor to enable easy collection of the biomarkers, or having biomarkers dispensed from the umbilical cord sensor by forcing air or another fluid through the microneedles.

As discussed herein, the disclosed umbilical cord sensors provide valuable real-time information during the birthing process in that it can be used to help assess the ideal time to clamp and cut an umbilical cord on a case-by-case basis. Presently decisions about when to cut the umbilical cord are not physiologically based. The umbilical cord sensors allow for any number of biomarkers, data, and other information associated with the respective umbilical cord sensor to be monitored, analyzed, studied, and/or otherwise used for various medical assessments, both in real-time and for purposes of providing information that can be utilized later (for that particular baby or for studies related to babies more generally). For example, the biomarkers and other data and information can be used to provide early detection of a disease in the newborn baby. Alternatively, or additionally, the biomarkers and other data and information can provide disease indicators and/or information about the newborn baby's susceptibility to various diseases. Still further, the biomarkers and other data and information can provide information about how the newborn baby is developing (e.g., cognitively) and/or is likely to develop in childhood. Genotyping and epigenetics are two other areas that can be enhanced based on the biomarkers and other data and information sensed, measured, collected, etc. by the umbilical cord sensors disclosed herein. This information for an individual baby can be used collectively with similar data from other babies to help provide valuable information about babies more generally. The collective data can be used to assist in early disease detection, identification of disease indicators and disease susceptibility, predicting aspects of child development (e.g., cognitive development), genotyping, and epigenetics, among other uses. This assessment of medical conditions and/or monitoring of developments can have immediate and life-long impacts on the individual newborn baby and on the population of newborn babies as a whole. Further, it may be possible that the biomarkers and other data and information sensed can be used for the benefits of fetuses and/or mothers, such as assessing medical conditions and/or monitoring their development.

Non-limiting examples of biomarkers that can be monitored, and, in at least some instances, some non-limiting examples of purposes for monitoring such biomarkers, include: red blood cell count, lactate, and/or APR. Non-limiting examples of samples that can be collected for lab analysis include: cord blood and/or tissue for genotyping and/or epigenetics analysis, and/or microbiome on a surface of the umbilical cord. By sampling and otherwise assessing biomarkers associated with the umbilical cord, it allows for sampling of the baby to be achieved without harming the baby. It also allows for a good volume of blood of the baby to be obtained, again without harming the baby.

FIGS. 5A and 5B illustrate still another exemplary embodiment of an umbilical cord sensor 210. Similar to the outer and inner bodies 124, 126 of the sensor 110 of FIG. 4, the sensor 210 includes a C-shaped outer body 224 and a C-shaped, expandable inner body 226, each having a circumference that exceeds three-quarters of the circumference of the umbilical cord 100 received by a receiving channel 222 defined by the expandable inner body 226. The sensor 210 can also include a two-way valve 230 that is operable to selectively inflate and deflate the inner body 226, as described in greater detail in other embodiments above. Further, although not illustrated, the sensor 210 can include one or both of: (1) one or more biosensors, or sensing devices; or (2) one or more sampling features. Despite these sensing devices and sampling features not being illustrated, a person skilled in the art, in view of the present disclosures, will understand how such devices and/or features can be incorporated into the umbilical cord sensor 210 of FIGS. 5A and 5B.

The expandable inner body 226 of FIGS. 5A and 5B is in a second expanded configuration, in which the inner body 226 expands even further radially inward than in the first expanded configuration illustrated in counterpart sensors 10, 110 of FIGS. 2B and 4. More particularly, in the second expanded configuration, also referred to as a clamped configuration, the umbilical cord 100 is clamped, thereby cutting off blood flow through the arteries 102, 104 and vein 106 of the umbilical cord 100 and separating the newborn baby from the placenta. A person skilled in the art will appreciate how much the inner body 226 needs to be expanded to sufficiently clamp the umbilical cord 100 such that blood flow is terminated. This may be known and/or informed by one or more sensors and related feedback provided for by the umbilical cord sensor 210, as described herein. A volume of the inner body 226 in the second expanded configuration can be, by way of non-limiting example, approximately in the range of about 1 milliliter to about 3 milliliters, and in some embodiments it can be about 1.75 milliliters.

In some embodiments, such as shown in FIG. 5B, a cutting device or feature can be incorporated as part of the umbilical cord sensor 210. This can be, for example, a component configured to provide a laser cut, illustrated as laser cutter 250 in FIG. 5B, and/or a knife or sharp edge disposed within the outer body 224 and configured to be deployed to cut the cord 100. Alternatively, or additionally, cutting the umbilical cord 100 after the umbilical cord sensor 210 clamps the umbilical cord 100 can be performed by an outside cutting device, such as a laser cut, a knife, or scissors. Whether the cutting device(s) and/or feature(s) is incorporated into the umbilical cord sensor 200, or is a component separate from the umbilical cord sensor 200, the cutting device(s) and/or feature(s) can be deployed based on feedback from the umbilical cord sensor 200 (such as feedback from sensors and sampling features). The deployment of the cutting device(s) and/or feature(s) can be automated in a manner similar to the two-way valve 30, 130, 230 described above in that the one or more sensors (e.g., biosensors), sampling features, and/or other data gathering components or information more generally can provide the necessary feedback to inform the umbilical cord sensor 210 that the umbilical cord 100 is ready to be cut, subsequently deploying the cutting device(s) and/or feature(s), as shown the laser cutter 250.

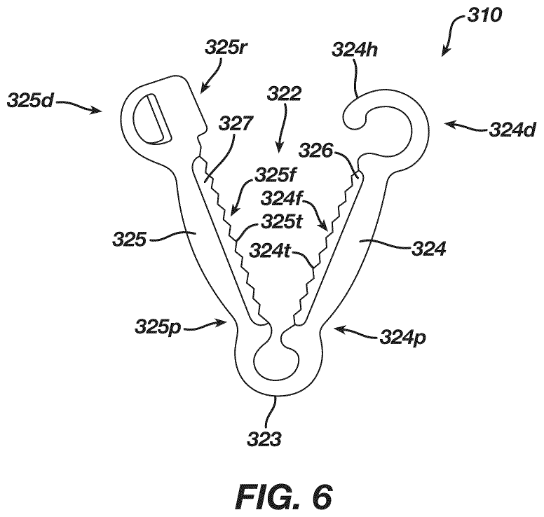

An embodiment in which aspects of the present disclosure are incorporated into a more traditional umbilical cord clamp is illustrated in FIG. 6, providing for an alternative embodiment of an umbilical cord sensor 310. As shown the sensor 310 includes two opposed arms, identified as a first arm 324 and a second arm 325, pivotally coupled to each other at a joint 323. One or both arms 324, 325 can move with respect to the other, the two arms 324, 325 defining a receiving channel 322 for receiving an umbilical cord therein. Proximal ends 324p, 325p of the respective arms 324, 325 can extend from the joint 323 and distal ends 324d, 325d of the respective arms 324, 325 can be configured to couple to together to hold one arm 324 with respect to the other 325. In the illustrated end, the distal end 324d of the first arm 324 includes a male coupling mechanism, as shown a hook 324h, and the distal end 325d of the second arm 325 includes a female coupling mechanism, as shown a receiver 325r. When the hook 324h mates to the receiver 325r, the sensor 310 is in a clamped position. Disposed on inner faces 324f, 325f of the first and second arms 324, 325, respectively, can be a plurality of teeth, as shown first and second teeth 324t, 325t. In other embodiments, the teeth 324t, 325t can be associated with another portion of the arm 324, 325, such as the equivalent of an outer body. When the sensor 310 is in the fixed, clamped position, the teeth 324t, 325t can exert a force on an umbilical cord received therebetween, thereby clamping the cord to cut-off blood flow therethrough. In some embodiments the teeth 324t, 325t can be part of an expandable inner body 326, 327, which can allow for an alternative way(s) by which the amount of force supplied during clamping can be controlled. The expandable inner body 326, 327 can be controlled in manners known to those skilled in the art, in view of the present disclosure. For example, a two-way valve (not shown) can be provided to selectively expand and contract the expandable inner body 326, 327. In embodiments that include an expandable inner body 326, 327, remaining portions of the first and second arms 324, 325 can be described as an outer body. Features as described with respect to other embodiments related to inner and outer bodies can be applicable to the present sensor 310, unless it is clear to a person skilled in the art that such features are not transferable to a sensor that is more akin to a traditional umbilical cord clamp.

Although not illustrated, the sensor 310 can include one or both of: (1) one or more biosensors, or sensing devices; or (2) one or more sampling features. Despite these sensing devices and sampling features not being illustrated, a person skilled in the art, in view of the present disclosures, will understand how such devices and/or features can be incorporated into the umbilical cord sensor 310 of FIG. 6. The sensing devices and/or sampling features can be included with umbilical cord sensors that include outer bodies 324, 325 and expandable inner bodies 326, 327, as shown, or in more traditional umbilical cord clamps that include more of a singular structure without an expandable inner body (i.e., just the outer body) 324, 325. Likewise, although not illustrated, one or more cutting devices and/or features can be incorporated into, or used with, the sensor 310 of FIG. 6, based, at least in part, on the disclosures provided above with respect to the sensor 210 of FIG. 5B. For example, a laser cutter and/or a knife or sharp edge can be disposed within some portion of the outer body 324, 325 and can be configured to be deployed to cut the cord in manner similar to those discussed above (e.g., based on manual actuation, based on automated actuation, either of which can be informed by feedback from one or more sensors or the like).

In use, and with reference in at least some instances to the sensor 10 of FIGS. 1-2B, any of the sensors provided for herein (e.g., the sensor 10) or otherwise derivable from the present disclosures can be positioned onto or otherwise disposed on an umbilical cord 100 after the umbilical cord exits the vagina. Alternatively, a sensor can be positioned onto or otherwise disposed on the umbilical cord while it is still located in the mother's body, for example by passing it through the vagina and into the mother's body for placement on the umbilical cord. Earlier placement can allow for intrauterine and/or intrapartum monitoring, including but not limited to identifying damage and/or other complications during the pregnancy and/or deliver.

Once the sensor 10 is positioned at the desired location with respect to the umbilical cord 100, the inner body 26 can be expanded to the first expanded configuration, for example by operating the two-valve 30, thereby allowing the inner surface 26s of the inner body 26 to engage the umbilical cord 100, as shown in FIG. 2B. The sensors and/or sampling devices (e.g., the biosensor 40) associated with the umbilical cord sensor 10 can then be operated to sense and/or measure one or more parameters associated with one or more biomarkers of the umbilical cord 100 and/or blood passing through the umbilical cord 100. Alternatively, or additionally, the sensors and/or sampling devices (e.g., the biosensor 40) can obtain a sample of one or more biomarkers associated with the umbilical cord 100 and/or blood passing through the umbilical cord 100. The biomarkers can be detected, sensed, measured, and/or collected from the outside of the umbilical cord 100 and/or from within the umbilical cord 100, including from within the umbilical cord arteries 102, 104 and vein 106. This may include, for example, drawing blood from inside the umbilical cord 100.

The sensed and/or measured parameters, as well as the collected samples, can in turn be used in a variety of contexts. In real-time that information can help inform when to clamp and/or cut the umbilical cord 100. In conjunction with the same, the expandable inner body 26 can be further expanded to clamp the umbilical cord 100 and/or one or more cutting devices and/or features can be operated to cut the umbilical cord 100. Operation of the clamping and/or cutting features can be done manually in response to the sensor 100 and/or measured parameters and/or collected samples, or automated based on feedback controls implemented in conjunction with the umbilical cord sensor 10. Alternatively, or additionally, the sensed and/or measured parameters, as well as the collected samples, can be used to make various assessments that may be valuable to know soon after the baby is born and/or later as the data and information can concern anticipated developments for the baby. This includes but is not limited to monitoring for early detection of diseases, disease indicators, and/or disease susceptibility, as well as evaluating child development (e.g., cognitive development), genotyping, and/or epigenetics, among other uses.

Once the umbilical cord sensor 10 is no longer needed, it can be removed from the umbilical cord 100. This can be done, for example, by operating the two-way valve 30 to at least partially deflate the expandable inner body 26, thereby allowing the umbilical cord sensor 10 to be moved radially away from the umbilical cord 100. The timing of removal may vary. In some instances, it may be shortly after the baby is born (e.g., approximately in the range of about one minute to five minutes after the baby is born) or later (e.g., up to at least a few hours after the baby is born, such as one hour, or possibly even longer).

The umbilical cord sensors of the present disclosure can be designed to be disposed of after a single use, or they can be designed to be used multiple times. In either case, however, the sensors can be reconditioned for reuse after at least one use. Reconditioning can include any combination of the steps of disassembly of the sensors, followed by cleaning or replacement of particular pieces, and subsequent reassembly. In particular, the sensors can be disassembled, and any number of the particular pieces or parts of the sensors can be selectively replaced or removed in any combination. Upon cleaning and/or replacement of particular parts, the sensors can be reassembled for subsequent use either at a reconditioning facility, or by a surgical team immediately prior to a surgical procedure. Those skilled in the art will appreciate that reconditioning of a sensor can utilize a variety of techniques for disassembly, cleaning/replacement, and reassembly. Use of such techniques, and the resulting reconditioned sensor, are all within the scope of the present application.

Preferably, the sensors described herein will be processed before use. First, a new or used sensor is obtained and if necessary cleaned. The sensor can then be sterilized. In one sterilization technique, the sensor is placed in a closed and sealed container, such as a plastic or TYVEK® bag. The container and its contents are then placed in a field of radiation that can penetrate the container, such as gamma radiation, x-rays, or high-energy electrons. The radiation kills bacteria on the sensor and in the container. The sterilized sensor can then be stored in the sterile container. The sealed container keeps the sensor sterile until it is opened in the medical facility.

It is preferred that the sensor is sterilized. This can be done by any number of ways known to those skilled in the art including beta or gamma radiation, ethylene oxide, or steam.

The above-provided description of a method of using the umbilical cord sensor provides one exemplary, non-limiting example of device use. Other methods are possible. For example, in some instances, it may be desirable to use more than one sensor at a time, and the sensors can, but do not have to be, of different constructions or configurations. Further, while the present disclosure focuses on an umbilical cord sensor, a person skilled in the art will recognize the teachings herein can be applied to other anatomies. By way of non-limiting example, the disclosures herein may be able to be applied to monitor the placenta. Accordingly, when monitoring anatomy like the placenta, useful information may be obtained about the nine months in utero that can provide valuable information as it relates to detection of diseases, disease indicators, and/or disease susceptibility, as well as evaluating child development (e.g., cognitive development), genotyping, and/or epigenetics, among other information and assessments.

One skilled in the art will appreciate further features and advantages of the disclosure based on the above-described embodiments. Accordingly, the disclosure is not to be limited by what has been particularly shown and described, except as indicated by the appended claims. All publications and references cited herein are expressly incorporated herein by reference in their entirety.

1. An umbilical cord sensor, comprising:
an outer body configured to be disposed at least partially around an umbilical cord;
an expandable inner body disposed within the outer body and configured to be disposed at least partially around and in contact with the umbilical cord;
a receiving channel defined by the expandable inner body, the receiving channel being configured to receive the umbilical cord; and
at least one of:
one or more biosensors structured and arranged to at least one of sense or measure one or more parameters associated with at least one of the umbilical cord or blood passing through the umbilical cord around which the outer body is at least partially disposed; or
one or more sampling features structured and arranged to obtain one or more biomarkers associated with at least one of the umbilical cord or blood passing through the umbilical cord around which the outer body is at least partially disposed.

2. The umbilical cord sensor of claim 1, further comprising:
a two-way valve associated with the expandable inner body to allow fluid communication between the expandable inner body and a fluid source such that a volume of the expandable inner body can be selectively expanded and contracted.

3. The umbilical cord sensor of claim 1, wherein the umbilical cord sensor comprises the one or more sampling features, the one or more sampling features further comprising:
at least one microchannel associated with the expandable inner body, in communication with at least one of the one or more biosensors or one or more separately disposed biosensors such that one or more biomarkers associated with an outer surface of the umbilical cord can be analyzed by the respective one or more biosensors of the umbilical cord sensor or the one or more separately disposed biosensors.

4. The umbilical cord sensor of any of claim 1, wherein the umbilical cord sensor comprises the one or more sampling features, the one or more sampling features further comprising:
at least one microneedle associated with the expandable inner body and configured to penetrate the umbilical cord to draw blood from the umbilical cord, the at least one microneedle being in communication with at least one of the one or more biosensors or one or more separately disposed biosensors such that one or more biomarkers associated with the drawn blood can be analyzed by the respective one or more biosensors of the umbilical cord sensor or the one or more separately disposed biosensors.

5. The umbilical cord sensor of any of claim 1, wherein the umbilical cord sensor comprises the one or more biosensors, the one or more biosensors further comprising:
at least one pulse oximeter associated with the expandable inner body and configured to sense or measure one or more biomarkers associated with at least one of the umbilical cord or blood passing through the umbilical cord around which the outer body is at least partially disposed.

6. The umbilical cord sensor of any of claim 1, further comprising:
at least one pressure sensor associated with the expandable inner body and configured to assess at least one of an amount of pressure being exerted by the expandable inner body on the umbilical cord or an amount of pressure being experienced by the umbilical cord.

7. The umbilical cord sensor of any of claim 1, further comprising:
at least one cutting device associated with at least one of the outer body and the expandable inner body.

8. The umbilical cord sensor of any of claim 1, wherein the outer body further comprises:
a first arm;
a second arm opposed to the first arm;
first teeth associated with at least one of the first arm or a portion of the expandable inner body disposed within the first arm;
second teeth associated with at least one of the second arm or a portion of the expandable inner body disposed within the second arm, the first and second teeth being opposed to each other and configured to clamp the umbilical cord around which the outer body is at least partially disposed.

9. The umbilical cord sensor of any of claim 1, wherein the outer body is configured to be disposed around a majority of a circumference of a cross-section of a portion of the umbilical cord to which the umbilical cord sensor is disposed.

10. A method of monitoring one or more biomarkers, comprising:
disposing an umbilical cord sensor on an umbilical cord such that the sensor is disposed around at least a portion of the umbilical cord;
at least one of:
sensing or measuring one or more parameters associated with one or more biomarkers of at least one of the umbilical cord or blood passing through the umbilical cord around which the umbilical cord sensor is disposed; or
obtaining a sample of one or more biomarkers associated with at least one of the umbilical cord or blood passing through the umbilical cord around which the umbilical cord is disposed.

11. The method of claim 10, further comprising:
expanding an expandable body of the umbilical cord sensor to assist in securing the umbilical cord sensor to the umbilical cord.

12. The method of claim 11, wherein expanding an expandable body of the umbilical cord sensor to assist in securing the umbilical cord sensor to the umbilical cord further comprises:
using at least one pressure sensor associated with the umbilical cord sensor to assess at least one of an amount of pressure being exerted by the expandable body on the umbilical cord or an amount of pressure being experienced by the umbilical cord.

13. The method of claim 11, wherein expanding an expandable body of the umbilical cord sensor to assist in securing the umbilical cord sensor to the umbilical cord further comprises:
operating a two-way valve to expand the expandable body

14. The method of any of claim 10, wherein the method comprises obtaining a sample of one or more biomarkers associated with at least one of the umbilical cord or blood passing through the umbilical cord around which the umbilical cord is disposed, such sample obtaining further comprising:
obtaining one or more biomarkers associated with an outer surface of the umbilical cord.

15. The method of any of claim 10, wherein the method comprises obtaining a sample of one or more biomarkers associated with at least one of the umbilical cord or blood passing through the umbilical cord around which the umbilical cord is disposed, such sample obtaining further comprising:
drawing blood from the umbilical cord sensor.

16. The method of any of claim 10, wherein the method comprises sensing or measuring one or more parameters associated with one or more biomarkers of at least one of the umbilical cord or blood passing through the umbilical cord around which the umbilical cord sensor is disposed, such sensing or measuring further comprising:
sensing or measuring using at least one pulse oximeter associated with the umbilical cord sensor.

17. The method of any of claim 10, further comprising:
clamping the umbilical cord with the umbilical cord sensor.

18. The method of any of claim 10, further comprising:
cutting the umbilical cord using a cutting device of the umbilical cord sensor.

19. The method of any of claim 10, wherein disposing an umbilical cord sensor on an umbilical cord further comprises:
disposing the umbilical cord sensor around a majority of a circumference of a cross-section of a portion of the umbilical cord to which the umbilical cord sensor is disposed.

20. The method of any of claim 10, wherein the one or more biomarkers are associated with at least one of the umbilical cord or a placenta.

21. The method of any of claim 10, further comprising:
assessing a medical condition of at least one of a fetus, a baby, or a mother based on the one or more biomarkers.

22. The method of any of claim 10, further comprising:
monitoring development of at least one of a fetus, a baby, or a mother based on the one or more biomarkers.
